(19) United States
(12) Patent Application Publication
(10) Pub. No.: US 2012/0232582 A1
(43) Pub. Date: Sep. 13, 2012

(54) VASCULAR CLOSURE PLUG WITH PROXIMAL CAP AND METHODS OF USE

(75) Inventors: Kristopher Vietmeier, Monticello, MN (US); Scott Schewe, Eden Prairie, MN (US); David Rolf, Eden Prairie, MN (US)

(73) Assignee: BOSTON SCIENTIFIC SCIMED, INC., Maple Grove, MN (US)

(21) Appl. No.: 13/416,053

(22) Filed: Mar. 9, 2012

Related U.S. Application Data
(60) Provisional application No. 61/451,833, filed on Mar. 11, 2011.

(51) Int. Cl. A61B 17/08

(52) U.S. Cl. 606/213

(57) ABSTRACT

The disclosure pertains to a vascular closure plug having a reinforced proximal end and methods of use therefor. The reinforcement may comprise a proximal force distributing member and a plurality of plug restraining members thereby tending to limit tearing or spreading of at least the proximal end of the vascular closure plug. In some embodiments, the reinforcement may also comprise a distal force distributing member and a plurality of plug restraining members, a circumferential collar, and/or a supplemental force distributing member.

### RELATED APPLICATIONS

This application claims priority to U.S. Provisional Application No. 61/451,833, filed Mar. 11, 2011.

### BACKGROUND

In many medical procedures, such as, for example, balloon angioplasty and the like, an opening can be created in a blood vessel or arteriotomy to allow for the insertion of various medical devices which can be navigated through the blood vessel to the site to be treated. For example, a guidewire may first be inserted through a tissue tract created between the skin, or the epidermis, of the patient down through the subcutaneous tissue and into the opening formed in the blood vessel. The guidewire is then navigated through the blood vessel to the site of the occlusion or other treatment site. Once the guidewire is in place, an introducer sheath can be inserted over the guide wire to form a wider, more easily accessible, tract between the epidermis and the opening into the blood vessel. The appropriate medical device can then be introduced over the guidewire through the introducer sheath and then up the blood vessel to the site of the occlusion or other treatment site.

Once the procedure is completed, the medical devices or other equipment introduced into the vessel can be retracted through the blood vessel, out the opening in the blood vessel wall, and out through the tissue tract to be removed from the body. The physician or other medical technician is presented with the challenge of trying to close the opening in the blood vessel and/or the tissue tract formed in the epidermis and subcutaneous tissue. A number of different device structures, assemblies, and methods are known for closing the opening in the blood vessel and/or tissue tract, each having certain advantages and disadvantages. However, there is an ongoing need to provide new and improved device structures, assemblies, and/or methods for closing and/or sealing the opening in the blood vessel and/or tissue tract.

### SUMMARY

This disclosure pertains to a vascular or tissue tract closure plug having a reinforced proximal cap which minimizes distortions during vascular closure plug deployment by providing a more uniform distribution of axial forces across the top of the plug during positioning and compression/expansion to a desired shape.

In one aspect, this disclosure relates to a device for sealing an opening in a vessel wall and/or tissue tract, the device comprising an expandable plug having a proximal end, a distal end, a central axis joining the proximal end and distal end, and at least one sidewall therebetween, said expandable plug being adapted to expand generally perpendicular to the central axis in response to axial compression and/or exposure to a liquid; a proximal force distributing member generally coextensive with the proximal end of the expandable plug prior to expansion; and a plurality of plug restraining members each having a proximal end attached directly or indirectly to the proximal force distributing member, distal ends, and lateral extents, wherein the plurality of plug restraining members extend generally parallel to the central axis prior to expansion of the expandable plug and the distal ends of the plurality of plug restraining members are displaced radially outward when the expandable plug is expanded.

In another aspect, this disclosure relates to a method of sealing an opening in a vessel wall and/or tissue tract comprising positioning an expandable plug within a tissue tract, said expandable plug being adapted to expand generally perpendicular to the central axis in response to axial compression and/or exposure to a liquid, a proximal force distributing member generally coextensive with the proximal end of the expandable plug, and a plurality of plug restraining members each having a proximal end attached directly or indirectly to the proximal force distributing member, a distal end, and a lateral extent, wherein the plurality of plug restraining members extend generally parallel to the central axis prior to expansion of the expandable plug and the distal ends of the plurality of plug restraining members are displaced radially outward from the respective proximal ends of said plug restraining members when the expandable plug is expanded; compressing said expandable plug axially within the tissue tract thereby expanding at least a portion of the expandable plug radially; and exposing the expandable plug to a liquid, whereupon the expandable plug is substantially prevented from expanding axially in the proximal direction by the force distributing member and the plurality of plug restraining members.

### BRIEF DESCRIPTION OF DRAWINGS

FIG. 1A illustrates schematically an expandable plug deployed in a tissue tract adjacent to a puncture in a vessel wall.

FIG. 1B illustrates an idealized schematic representation of an expanded plug deployed within a tissue tract adjacent to a puncture in a vessel wall.

FIG. 1C illustrates schematically several failure modes which may occur when an expandable plug of the prior art is deployed in a tissue tract adjacent to a puncture in a vessel wall.

FIG. 2 illustrates an expandable plug of the present disclosure and an optional separate reinforcing member.

FIG. 3 illustrates an alternate expandable plug of the present disclosure.

FIG. 4 illustrates another embodiment of the expandable plug of the present disclosure.

FIG. 5 illustrates yet another embodiment of the expandable plug of the present disclosure.

FIG. 6 illustrates yet another embodiment of the expandable plug of the present disclosure.

FIG. 7 illustrates a plug of the present disclosure in a partially expanded state.

FIG. 8 illustrates an alternate plug of the present disclosure in a partially expanded state.

FIG. 9 illustrates another plug of the present disclosure in a partially expanded state.

### DETAILED DESCRIPTION

The following description should be read with reference to the drawings wherein like reference numerals indicate like elements throughout the several views. The drawings, which are not necessarily to scale, are not intended to limit the scope of the claimed invention. The detailed description and drawings illustrate example embodiments of the claimed invention.

All numbers are herein assumed to be modified by the term “about.” The recitation of numerical ranges by endpoints includes all numbers subsumed within that range (e.g., 1 to 5 includes 1, 1.5, 2, 2.75, 3, 3.80, 4, and 5). As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to select and effect such feature, structure, or characteristic in connection with other embodiments whether or not explicitly described in combination.

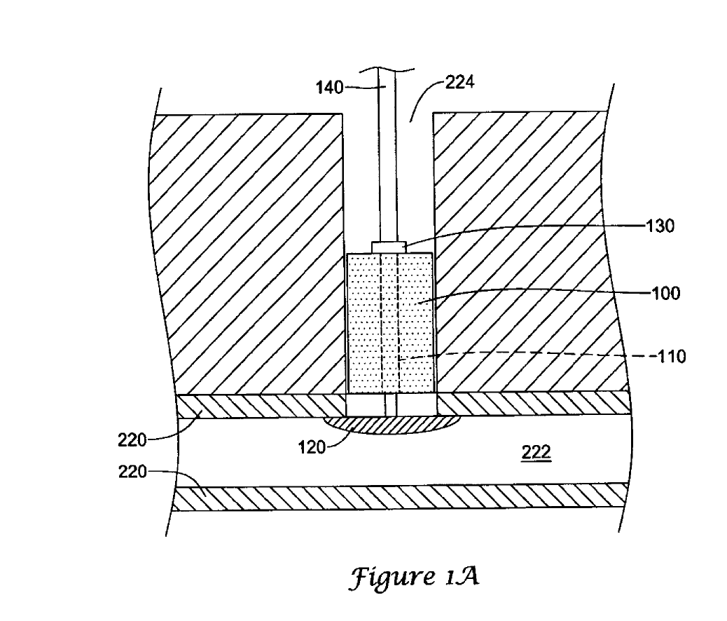

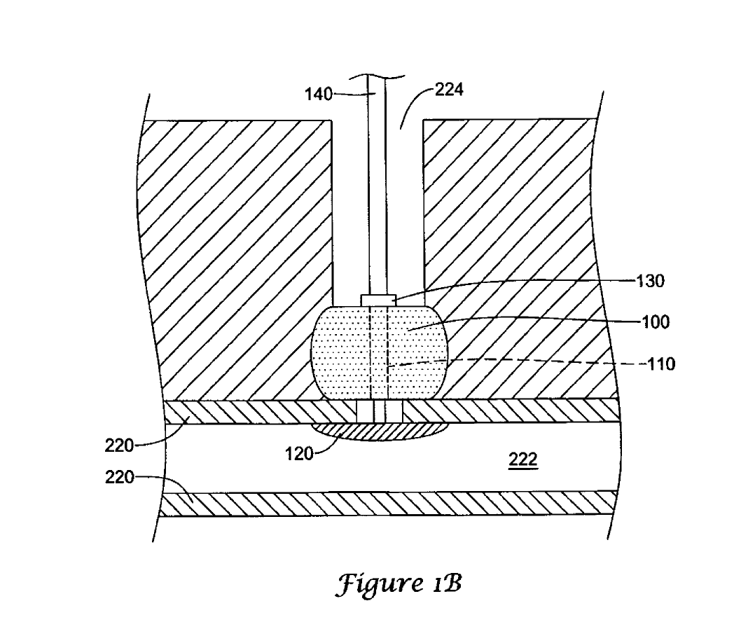

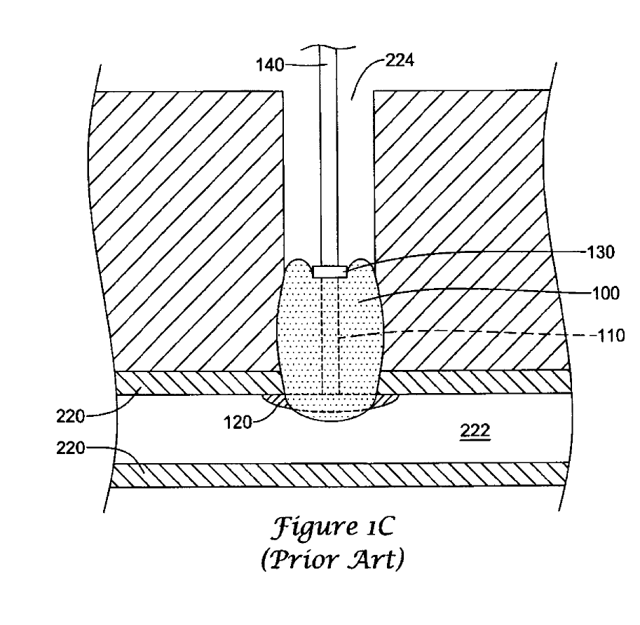

FIG. 1A, shows a representation of an expandable plug 100 initially deployed in a tissue tract 224 adjacent to a puncture in a vessel wall 220. The expandable plug 100 is secured within tissue tract 224 between an anchor 120 disposed in the vessel lumen 222 and a bead, button, or knot 130 which may be connected by an element 140. Element 140 may be any of the structures employed in the art for the purpose, for example one or more sutures, a shaft, or a tubular element. When element 140 is a tubular element, it may be used to pass additional elements into or out of the vessel or it may serve as a bleed back tube to aid in positioning the expandable plug 100. Element 140 is shown as extending through a bore 110 in expandable plug 100; however other arrangements are possible. The distal end of element 140 may be attached to anchor 120 directly or through a flexible coupling (not shown). Anchor 120 may be any of the anchoring means known in the art and may include rods, disks, clips, stitches, or other elements known for that purpose. The anchor 120, like other elements of the plug 100 and the associated components of the inventive device often are formed from biocompatible and bioabsorbable materials; however this is not necessarily the case as it may be desirable for some elements to persist for extended periods of time or to be removed before biocompatibility becomes a significant issue.

FIG. 1B illustrates a deployment of an expandable plug 100 deployed in its expanded state within tissue tract 224. The expandable plug 100 has been axially compressed by sliding a bead, button, or knot 130 along element 140. In addition or in the alternative, the expandable plug 100 may have been at least partially expanded by exposing the expandable plug 100 to a liquid. The expandable plug 100 has occluded the puncture in vessel wall 220 and has expanded laterally to seal the tissue tract 224. It is often desirable for the expanded plug 100 to effectively seal the boundary between the exterior vessel wall 220 and the tissue surrounding tissue tract 224 to prevent lateral leakage.

FIG. 1C illustrates common deployment failure modes encountered during the use of expandable plugs 100 of the prior art. For example, the plug may fail to expand laterally to the desired extent, in part because of a failure to remain contained between the exterior vessel wall 220 and the bead, button, or knot 130. As illustrated, a distal portion of the expandable plug 100 has extruded into the vessel through the puncture. In some circumstances, the bead, button, or knot 130 may be insufficient to keep the proximal end of the expandable plug confined. In more extreme instances, the bead, button, or knot 130 and/or the one or more sutures, shaft, or tubular element 140 may tear through the expandable plug 100 allowing a significant expanded volume of the expandable plug 100 to avoid lateral expansion which may result in diminished sealing of the tissue tract 224.

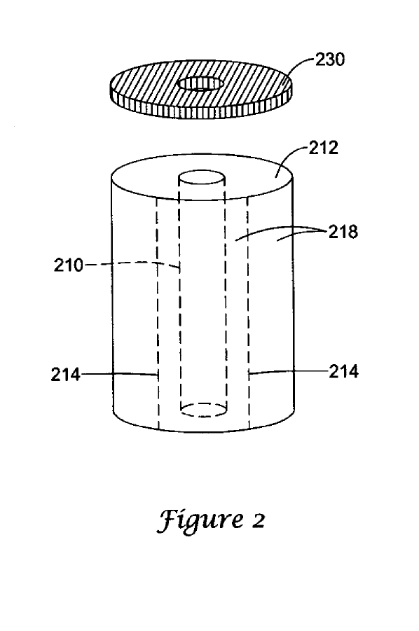

FIG. 2 illustrates a representative, nonlimiting embodiment of the present disclosure in which an expandable plug 200 is partially surrounded by a proximal force distributing member 212 disposed adjacent to the proximal end of the expandable plug 200 and a plurality of plug restraining members 218 which are disposed along the sidewall of a generally cylindrical expandable plug 200. In some embodiments, plug restraining members 218 collectively substantially cover a proximal end region of the at least one sidewall of the expandable plug 200. In other embodiments, plug restraining members 218 cover only a proximal end region of the at least one sidewall of the expandable plug 200. In yet other embodiments, there are gaps between the plug restraining members 218.

The proximal force distributing member 212 and plug restraining members 218 may be fabricated from commonly employed bioabsorbable materials including, but not limited to polylactic acid, polylactic-co-glycolic acid, polyglycolic acid, polycaprolactone, and the like. In some embodiments, in which only brief containment is required, faster degrading/dissolving/absorbing materials may be used or may be included in the formulation. Such materials may include, for example a biodegradable polymer blended with sugar esters such as sucrose octaacetate, sucrose acetate isobuterate, β-D-galactose pentaacetate, β-D-glucose pentaacetate, α-D(+)-glucose pentaacetate, and the like. Some formulations may beneficially include plasticizers such as acetyl tributyl citrate, glycerol and its esters, sorbitol, triacetin, and the like. Other embodiments may beneficially include soluble fillers. In some embodiments, the materials may be somewhat tacky to better remain in position relative to the expandable plug 200 during deployment. In other embodiments, the materials may be selected to be somewhat lubricious to facilitate transport within an introducer sheath and/or a tissue tract.

The proximal force distributing member 212 and plug restraining members 218 may be fabricated by molding, extrusion, solvent casting, and the like. In some embodiments, the proximal force distributing member 212 and plug restraining members 218 may be formed separately and then joined. The proximal force distributing member 212 and plug restraining members 218 are not necessarily formed of the same materials. In other embodiments, the proximal force distributing member 212 and plug restraining members 218 may be integrally formed. In some embodiments, the proximal force distributing member 212 and the plug restraining members 218 are joined along a weakened line which facilitates a hinge-like fold at the transition therebetween.

The expandable plug 200 may be any of those employed in the art. For example, expandable plug 200 may comprise a sponge-like material (e.g., naturally occurring collagens, synthetic collagens, or other biologically resorbable sponge-like material), a foam, or a fibrous woven or nonwoven material, and may be configured in any shape to facilitate sealing the puncture in vessel wall 220 and tissue tract 224. The expandable plug 200 may initially be flexible or somewhat rigid. In some embodiments the expandable plug 200 may comprise a hemostatic foam. Plug 200 may be a material of uniform composition or may include layers and/or regions of different composition. The expandable plug 200 may also include a hemostatic agent, such as a tissue thromboplastin, to accelerate local hemostasis. In some embodiments, the expandable plug 200 may comprise a hemostatic foam. In other embodiments, the expandable plug may comprise a substantially axial central bore connecting the proximal end of the expandable plug to the distal end of the expandable plug.

As illustrated, proximal force distributing member 212 is generally coextensive with the proximal end of the expandable plug 200; however it will be appreciated that the proximal force distributing member 212 may be larger than or smaller than the proximal end of the expandable plug 200 and may optionally include holes or other apertures. Such holes may provide access to an interior bore 210 and/or may provide fluid access to the expandable plug.

As illustrated in FIG. 2, plug restraining members 218 are fixedly attached at their proximal ends to proximal force distributing member 212 and cover substantially the entire sidewall of expandable plug 200 (not visible in FIG. 2). The points of attachment between restraining members 218 and proximal force distributing member 212 may optionally be scored, relieved, perforated, or otherwise modified to ensure that the plug restraining members 218 are hinged to allow relatively free movement as the expandable plug 200 expands.

In some embodiments, the plug restraining members 218 may be perforated along adjacent edges 214 to an extent which will allow the plug restraining members 218 to separate as the expandable plug 200 begins to expand upon compression and/or exposure to liquid. Such perforations may provide a degree of integrity to the unified proximal force distributing member 212 and plug restraining members 218 to facilitate assembly of the device prior to insertion into the tissue tract 224 or within the tissue tract 224.

In other embodiments, the proximal force distributing member 212, plug restraining members 218, and expandable plug 200 may be integrally formed. In yet other embodiments, the proximal force distributing member 212 and plug restraining members 218 may be formed as a coating on the expandable plug 200 and optionally may be slit or perforated after formation.

As illustrated in FIG. 2, the device may optionally include a supplemental force distributing member 230 which also may optionally include holes or other apertures. Such holes may provide access to an interior bore 210 and/or may provide fluid access to the expandable plug. When present, the supplemental force distributing member 230 may be a separate element of the device or it may be attached to the proximal force distributing member 212. It may be integrally formed with the proximal force distributing member 212. In some embodiments, the supplemental force distributing member 230 may be formed as a thickened region of the proximal force distributing member 212. Although the illustrated supplemental force distributing member 230 is coextensive with proximal force distributing member 212, it may be larger than or smaller than the proximal force distributing member 212. The shape of the supplemental force distributing member 230 may be the same or different from that of the proximal force distributing member 212. The composition of the supplemental force distributing member 230 may be the same or different from that of the proximal force distributing member 212. In some embodiments, the supplemental force distributing member 230 may be designed to dissolve or be bioabsorbed more rapidly than the proximal force distributing member 212 and/or the plug restraining members 218.

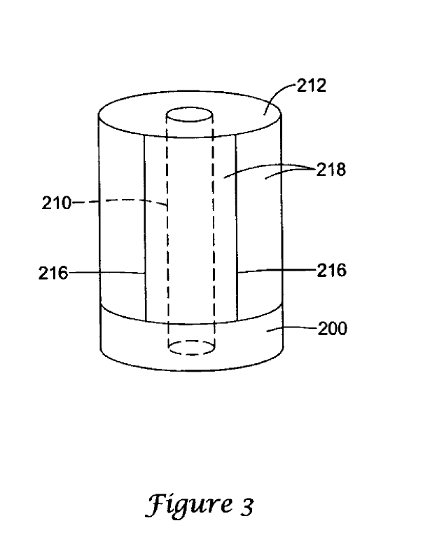

In the embodiment of FIG. 3, the plug restraining members 218 cover only a proximal portion of the expandable plug 200. The plug restraining members 218 are slit along parting lines 216. In some embodiments, there may be gaps between the plug restraining members 218. Although not illustrated, one of ordinary skill in the art will appreciate that perforations, slits, or gaps 216 between the plug restraining members 218 need not be linear or generally parallel to the axis of the expandable plug 200. The perforations, slits, or gaps 216 may, for example, take the form of a partial helix and/or optionally may be nonlinear in the form of a zigzag or sinusoidal or other curve.

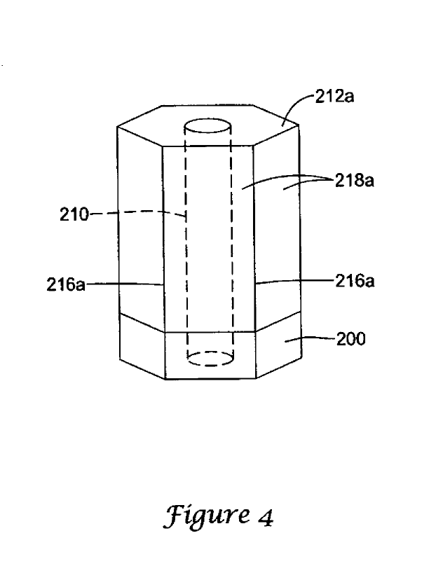

The embodiment of FIG. 4 illustrates that the expandable plug need not have the general form of a cylinder, but may assume a prismatic form having any number of facets. Although six sidewall facets are illustrated, there may be fewer or more facets. For example, there may be 3, 4, 5, 7, 8, or more facets. As in the earlier figures, the plug restraining members 218a may be separated by perforations, slits, or gaps 216a which may take on the forms described above.

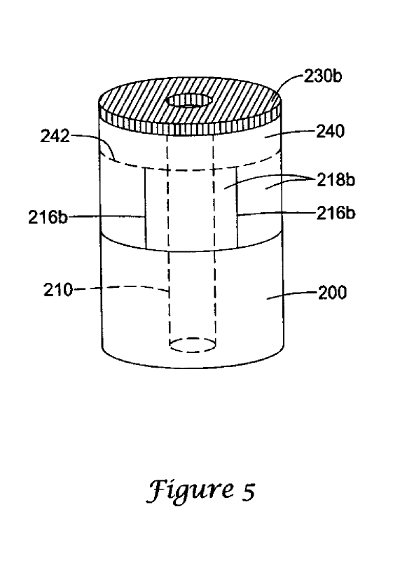

FIG. 5 illustrates further variations in the structural elements which may be combined in devices of the present disclosure. For example, the device of FIG. 5 includes a collar 240 fixedly attached between plug restraining members 218b and proximal force distributing member 212b (not shown). In some embodiments, the proximal force distributing member 212b may be supplemented or entirely replaced by supplemental force distributing member 230b. In those embodiments which include a collar, such as collar 240, the plug restraining members 218b may be fixedly attached along line 242 to the collar 240 in any of the manners described above for the attachment of plug restraining members 218 to proximal force distributing member 212. Similarly, a collar may be faceted to conform to a prismatic plug such as that illustrated in FIG. 4. In embodiments which include a collar, the plug restraining members 218b may cover substantially the entire remaining side surface(s) or may only cover a region of expandable plug 200 adjacent to the collar. As described above, plug restraining members 218b may take on various shapes and initially may be at least intermittently joined along mutual edges or may be separated from each other.

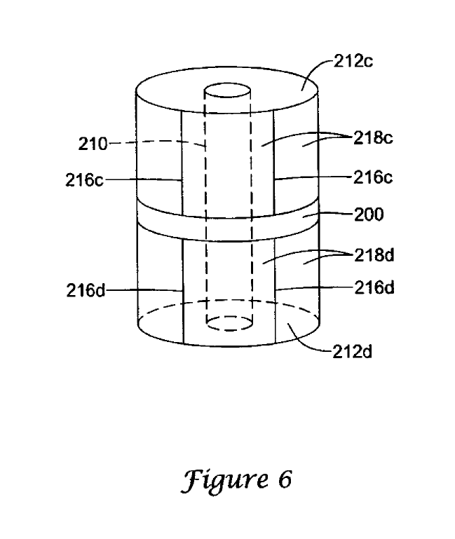

FIG. 6 illustrates an embodiment in which the distal end of an expandable plug 200 is provided with an additional force distribution member 212d and fixedly attached plug restraining members 218d. This distal portion of the device may share any of the characteristics of the proximal devices described above including the presence of an optional distal collar (not shown) fixedly attached to the distal plug restraining members 218d. It is believed that distal elements may provide additional control over the expansion of expandable plug 200 and further may tend to inhibit entry of the expandable plug 200 into the vessel during expansion. The composition and construction of distal elements may be the same or different from the corresponding proximal elements in a given device. A device having these distal elements may comprise a plurality of distal plug restraining members 218b having a proximal end, a distal end, and a lateral extent, wherein the plurality of distal plug restraining members 218b extend generally parallel to the central axis prior to expansion of the expandable plug and the proximal ends of the plurality of distal plug restraining members are displaced radially outward from the respective distal ends of said distal plug restraining members when the expandable plug is expanded. Distal plug restraining members 218b may collectively substantially cover a distal end region of an at least one sidewall of the expandable plug 200. Although the distal and proximal structures have been illustrated as having a gap therebetween, the respective distal and proximal plug restraining members may contact each other or even overlap adjacent to a central portion of the sidewall of the expandable plug 200. In some embodiments, the respective free ends of the distal and proximal plug restraining members may partially interdigitate.

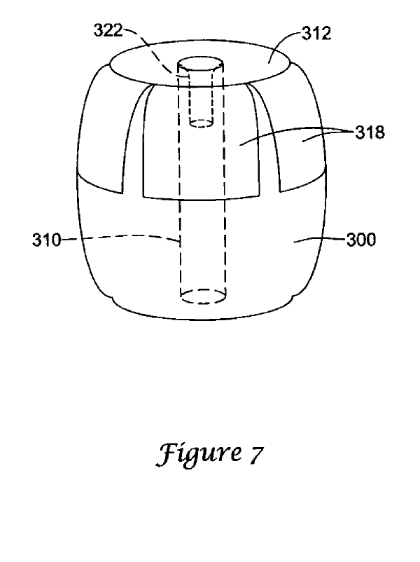

FIG. 7 illustrates schematically an embodiment of the present disclosure in a partially expanded condition similar to that which may occur during deployment within a tissue tract (not shown for clarity). As will be seen, the expandable plug 300 has expanded under the influence of a compressive force and/or has swollen upon exposure to a liquid. Plug restraining members 318 have separated and have tended to ride up onto the proximal end surface of the expandable plug 300 as their proximal ends have been forced radially outward by the expansion. Depending upon the circumstances of the expansion and the structure of the surrounding tissue, further expansion may occur with time.

The proximal force distributing member of the embodiment of FIG. 7 includes an optional tubular element 322 disposed within a bore 310 of expandable plug 300. The optional tubular element 322 may tend to maintain the orientation of the proximal force distribution member in a plane generally perpendicular to the axis of the expandable plug 300. In those embodiments which include a supplemental force distribution member, a tubular element may be attached to the supplemental force distribution member instead. In some embodiments a tubular member may be attached to both a proximal force distribution member and a supplemental force distribution member. In those embodiments which include the distal elements of, for example FIG. 6, a tubular member may be present within the distal end of the bore 310 and may be attached to the corresponding distal force distribution member.

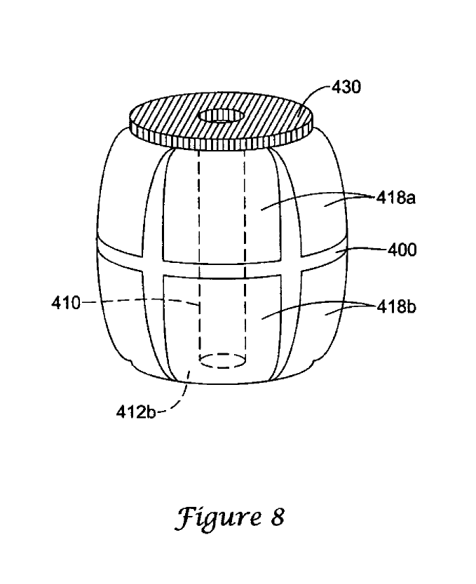

FIG. 8 illustrates schematically an alternate embodiment of the present disclosure in a partially expanded condition similar to that which may occur during deployment within a tissue tract (not shown for clarity). The embodiment of FIG. 8 includes distal force distribution member 412b and distal plug restraining members 418b. As with the proximal elements of FIG. 7, plug restraining members 418a and 418b have separated and have tended to occupy a portion of the respective end surfaces of the expandable plug 400 as their proximal ends have been forced radially outward by the expansion. Depending upon the circumstances of the expansion and the structure of the surrounding tissue, further expansion may occur.

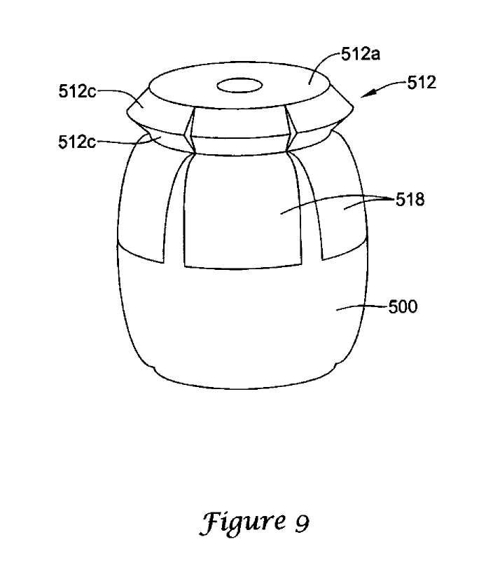

FIG. 9 illustrates somewhat schematically an alternate form of a proximal force distribution member 512 in a partially expanded condition. In this embodiment, a pair of force distribution members 512a and 512b (obscured by folding struts 512c) are separated by a plurality of folding struts 512c which allow the proximal force distribution member 512 to pass more readily through an introducer sheath and/or a tissue tract and yet deploy in a manner which imparts additional stiffness to the proximal force distribution member 512. In some embodiments, the proximal force distribution member 512 may have a greater radial extent than the expandable plug 500 with which it is associated.

In use, a device of the present disclosure may be inserted into a tissue tract adjacent to a vessel puncture or into a suitable lumen. The insertion may be accomplished with the assistance of any of the devices commonly employed for that purpose, such as guidewires, introducer sheaths, hydration chambers, and the like or may be directly inserted into the tissue tract or lumen. In some instances, the device will be used in conjunction with an anchor, such as anchor 120 of FIGS. 1-3, which may help to position the device and further may provide a degree of resistance to displacement of the distal end of the device during compression. When the device is deployed in a lumen, a supplemental force distribution member may be employed to provide a degree of resistance to displacement of the distal end of the device during compression. In the alternative, the proximal end may be held stationary and a supplemental force distribution member may be urged proximally to compress the device.

An optional bore (110, 210, 310) passing axially through the device may accommodate a guidewire, one or more sutures, a placement shaft or tube, or the like. A tube or shaft, such as element 140 of FIGS.1-3, positioned in such a bore (110, 210, 310) may provide additional control over positioning and the initial expansion of the device. In some embodiments, the insertion step may include pre or post hydration of the expandable plug 200 with, for example saline or other liquid.

As initially inserted, plug restraining members (218, 218a-d, 318, 418a-b, 518) will extend generally parallel to the surface(s) of the expandable plug (200, 300, 400, 500). In some embodiments, the components will be preassembled and inserted simultaneously; however portions of the device may be inserted sequentially. For example, a distal force distribution member 212d or 412b, with or without associated plug restraining members, may precede the plug into a tissue tract 210. Similarly, a supplemental force distribution member 230 or 512 may follow the expandable plug 200, 500 into a tissue tract 210. Upon mechanical compression or exposure to a liquid, the plug restraining members (218, 218a-d, 318, 418a-b, 518) will expand radially at their free ends to accommodate radial expansion of the expandable plug (200, 300, 400, 500). Within a tissue tract 210, the device is allowed to expand in response to exposure to liquid and/or upon being subjected to axial compression. In some embodiments, axial compression may be created by an externally supplied compressive force such as might be produced by advancing a pusher (not shown) against the proximal end of a device while holding the distal end stationary.

In other embodiments a component at the distal end of the device may be withdrawn relative to the proximal end of the device by withdrawing a suture, shaft, or tube similar to those represented by element 140 of FIGS. 1-3.

During expansion of the device, the expanding expandable plug is substantially prevented from expanding axially by the force distributing member(s) and/or any force directing and plug restraining members (218, 218a-d, 318, 418a-b, 518). As discussed above, the device may include a distal force distributing member (212d, 412b) and a plurality of distal plug restraining members (218d, 418b) which may be inserted before, or concurrently with, the expandable plug (200, 400).

### EXPERIMENTAL

The nonlimiting examples provided herein are intended to be generally indicative of the materials and associated properties which are believed to be desirable for the formation of the force distribution members, plug restraining members, supplemental force distribution members, and incidental components of the devices of the present disclosure, whether they are disposed about a proximal or a distal end of an expandable plug. It will be appreciated that some of these materials may be better suited for use in various fabrication methods.

Small quantities of the ingredients indicated in Table I were combined and melted in an aluminum weighing dish and allowed to cool. Once cool, the aluminum weighing dishes were folded and the properties and response to physical distortion noted. For selected samples, a test coupon having dimensions of 2 millimeters (mm) by 3 mm by 1 mm were submerged in a solution of TWEEN 20® (Sigma-Aldrich, Inc., St. Louis, Mo.) in phosphate buffered saline to estimate the relative rates of solubility in a liquid similar to bodily fluids.

| Composition | Observations | Solubility |
|---|---|---|
| 90% sucrose octaacetate, | Clear glass, tough, | Nearly all dissolved |
| 10% sucrose acetate isobuterate | bent >130° before cracking, | in 24 hours |
| | shattered | |
| 90% sucrose octaacetate, | Clear glass, soft, sticky | NA |
| 10% acetyl tributyl citrate | | |
| 95% sucrose octaacetate, | Flexible glass, slight tack, | NA |
| 5% triacetin | cracked after 140° bend | |
| 90% sucrose octaacetate | Clear glass | NA |
| 5% acetyl tributyl citrate, 5% triacetin | | |
| 90% sucrose octaacetate, 5% acetyl tributyl | Clear glass, flexible, | Nearly all dissolved |
| citrate, 5% sucrose acetate isobuterate | Some cracks | in 24 hours |
| 90% sucrose octaacetate, 5% sorbitol, | Clear glass, slight tack | NA |
| 5% triacetin | | |
| 50% α-D(+)-glucose pentaacetate, | Scratches with fingernail, | All dissolved |
| 50% β-D-glucose pentaacetate | not brittle, can chip, adheres | in 24 hours |
| | to pan | |
| 50% β-D-glucose pentaacetate, | Curlable, can shatter, | All dissolved |
| 50% β-D-galactose pentaacetate | stretches, tacky, strong | in 24 hours |
| 50% sucrose octaacetate, | Scratches with fingernail, | All dissolved |
| 50% α-D(+)-glucose pentaacetate | curls easily in pan, slight | in 24 hours |
| | tack, good adhesion | |
| 50% α-D(+)-glucose pentaacetate, | Clear glass, brittle, weak | All dissolved |
| 50% β-D-galactose pentaacetate | | in 24 hours |

The non-limiting materials characterized above should be viewed as illustrative of the range of components which may be combined to provide suitable materials for the fabrication of devices of this disclosure and to indicate approximate suitable ranges thereof.

Although the illustrative examples described above relate to sealing a tissue tract and an adjacent vessel wall puncture, applications in which tissue wall punctures or other lumens are to be sealed are also contemplated. In such an embodiment, the anchors 120 discussed above may be omitted or otherwise replaced by suitable elements.

Various modifications and alterations of this present disclosure will become apparent to those skilled in the art without departing from the scope and principles of this present disclosure, and it should be understood that this present disclosure is not to be unduly limited to the illustrative embodiments set forth hereinabove. All publications and patents mentioned are herein incorporated by reference to the same extent as if each individual publication or patent was specifically and individually indicated to be incorporated by reference.

What is claimed is:

1. A device for sealing an opening in a vessel wall and/or tissue tract, the device comprising:
an expandable plug having a proximal end, a distal end, a central axis joining the proximal end and distal end, and at least one sidewall therebetween, said expandable plug being adapted to expand generally perpendicular to the central axis in response to one or more of axial compression and exposure to a liquid;
a proximal force distributing member generally coextensive with the proximal end of the expandable plug prior to expansion; and
a plurality of plug restraining members each having a proximal end attached directly or indirectly to the proximal force distributing member, distal ends, and lateral extents,
wherein the plurality of plug restraining members extend generally parallel to the central axis prior to expansion of the expandable plug and the distal ends of the plurality of plug restraining members are displaced radially outward when the expandable plug is expanded.

2. The device of claim 1, wherein the plurality of plug restraining members collectively substantially cover a proximal end region of the at least one sidewall of the expandable plug.

3. The device of claim 1, wherein the plurality of plug restraining members are fixedly attached at their proximal ends to the proximal force distributing member.

4. The device of claim 1, wherein the plurality of plug restraining members are fixedly attached at their proximal ends to a circumferential collar disposed around the expandable plug and adjacent to the proximal end of the expandable plug.

5. The device of claim 4, wherein the circumferential collar is fixedly attached to the proximal force distributing member.

6. The device of claim 1, further comprising a distal force distributing member generally coextensive with the distal end of the expandable plug prior to expansion of the expandable plug.

7. The device of claim 6, further comprising a plurality of distal plug restraining members having a proximal end, a distal end, and a lateral extent,
wherein the plurality of distal plug restraining members extend generally parallel to the central axis prior to expansion of the expandable plug and the proximal ends of the plurality of distal plug restraining members are displaced radially outward from the respective distal ends of said distal plug restraining members when the expandable plug is expanded.

8. The device of claim 6, wherein the plurality of distal plug restraining members collectively substantially cover a distal end region of the at least one sidewall of the expandable plug.

9. The device of claim 6, wherein the plurality of distal plug restraining members are fixedly attached at their distal ends to a distal force distributing member.

10. The device of claim 6, wherein the plurality of distal plug restraining members are fixedly attached at their distal ends to a distal circumferential collar disposed around the expandable plug and adjacent to the distal end of the expandable plug.

11. The device of claim 10, wherein the distal circumferential collar is fixedly attached to the distal force distributing member.

12. The device of claim 1, wherein the expandable plug comprises hemostatic foam.

13. The device of claim 1, wherein the expandable plug comprises a multi-component layered hemostatic foam.

14. The device of claim 1, further comprising a substantially axial central bore connecting the proximal end of the expandable plug to the distal end of the expandable plug.

15. The device of claim 14, wherein the proximal force distributing member comprises a tubular member disposed within the bore.

16. The device of claim 1, wherein each of the components of the device is biocompatible and bioabsorbable.

17. The device of claim 1, wherein the proximal force distributing member comprises a separate supplemental force distributing member.

18. The device of claim 1, wherein the proximal force distributing member comprises an integral supplemental force distributing member.

19. A method of sealing an opening in a vessel wall and/or tissue tract comprising:
positioning an expandable plug within a tissue tract, said expandable plug being adapted to expand generally perpendicular to the central axis in response to one or more of axial compression and exposure to a liquid, a proximal force distributing member generally coextensive with the proximal end of the expandable plug, and a plurality of plug restraining members each having a proximal end attached directly or indirectly to the proximal force distributing member, a distal end, and a lateral extent,
wherein the plurality of plug restraining members extend generally parallel to the central axis prior to expansion of the expandable plug and the distal ends of the plurality of plug restraining members are displaced radially outward from the respective proximal ends of said plug restraining members when the expandable plug is expanded;
compressing said expandable plug axially within the tissue tract thereby expanding at least a portion of the expandable plug radially; and
exposing the expandable plug to a liquid,
whereupon the expandable plug is substantially prevented from expanding axially in the proximal direction by the force distributing member and the plurality of plug restraining members.

20. The method of claim 19, wherein the inserting step further comprises inserting a distal force distributing member and a plurality of distal plug restraining members into the tissue tract distal of the distal end of the expandable plug.
